(19) United States
(12) Patent Application Publication
(10) Pub. No.: US 2019/0209466 A1
(43) Pub. Date: Jul. 11, 2019

(54) OPHTHALMIC COMPOSITIONS

(71) Applicants: DEXCEL PHARMA TECHNOLOGIES LTD., OR-AKIVA (IL); YISSUM RESEARCH DEVELOPMENT COMPANY OF THE HEBREW UNIVERSITY OF JERUSALEM LTD., Jerusalem (IL)

(72) Inventors: Simon BENITA, Tel-Aviv (IL); Taher NASSAR, Tur'an Village (IL)

(73) Assignees: DEXCEL PHARMA TECHNOLOGIES LTD., OR-AKIVA (IL); YISSUM RESEARCH DEVELOPMENT COMPANY OF THE HEBREW UNIVERSITY OF JERUSALEM LTD., Jerusalem (IL)

(21) Appl. No.: 16/326,954

(22) PCT Filed: Sep. 3, 2017
(86) PCT No.: PCT/IL2017/050980

§ 371 Date: Feb. 21, 2019

Related U.S. Application Data
(60) Provisional application No. 62/383,725, filed on Sep. 6, 2016.

(51) Int. Cl. A61K 9/00; A61K 47/38; A61K 47/36; A61P 27/02; A61P 31/04; A61K 31/575; A61K 47/26; A61K 47/10; A61K 47/02; A61K 47/18

(52) U.S. Cl. CPC A61K 9/0048; A61K 47/38; A61K 47/36; A61P 27/02; A61P 31/04; A61K 47/186; A61K 47/26; A61K 47/10; A61K 47/02; A61K 47/18; A61K 31/575

(57) ABSTRACT

An ophthalmic composition useful for the treatment of bacterial eye infections is provided. The ophthalmic composition includes the suspension of particles of active anti-bacterial agent in a gelled matrix.

### TECHNICAL FIELD

Ophthalmic compositions, methods for their production and use thereof are disclosed.

### BACKGROUND

Eye infections can be caused by bacteria, viruses or fungi. Typically, eye infections are accompanied by various symptoms including pain, foreign body sensation, swelling of the eye lids, discharge from the eye, redness of the eye or eyelids, itching, photophobia, and blurred or decreased vision. More serious eye infections affect the entire eye area (periorbital cellulitis) or the lacrimal sacs (dacryocystitis) and when untreated, may ultimately lead to permanently impaired vision.

Treatment of eye infections induced by bacteria typically includes topical administration of eye drops, ointments, gels or creams. The active ingredient in the aforementioned formulations is selected from various antibiotics of different classes such as polypeptide antibiotic, aminoglycoside antibiotic, macrolide antibiotic, antifolate antibiotic, quinolone antibiotic, sulfa antibiotic, and tetracycline antibiotic.

A problem which is often encountered with liquid eye drop formulations is that active ingredient selection is based, at least partially, on its miscibility characteristics whereas other important characteristics are often compromised. In addition, the residence time of liquid eye drops in the eye is limited. Therefore, a need arises for introduction of improved liquid eye drops formulations and/or other ointments, gels or creams that could overcome the flaws of hitherto known liquid eye drops formulations.

In U.S. Pat. No. 6,255,299 Deleuran discloses a method of treating eye infections comprising applying an effective amount of an ophthalmic gel composition comprising about 1% w/v of fusidic acid in the form of particles having a particle size of between 2 and 5 μm suspended in an aqueous vehicle containing from 0.2 to 2% w/v of carboxyvinyl polymer, said composition having a viscosity of from 10 to about 20,000 cps at 25° C. measured on a RVT Brookfield Viscometer and a pH of from 5.0 to 6.5, said composition being applied as an eye drop into the fornix inferior of the infected eye one or two times daily.

In U.S. Pat. No. 6,174,524 Bawa et al. disclose an improved ophthalmic composition comprising xanthan gum, wherein the improvement comprises the composition having a total ionic strength of about 120 mM or less and the xanthan gum having an initial bound acetate content of at least about 4% and an initial bound pyruvate content of at least about 2.5%, provided that the composition does not contain locust bean gum.

In U.S. 2012/0270955 Chowhan et al. disclose a topical ophthalmic multi-dose aqueous composition comprising: a viscosity enhancing system comprised of: i) dissipation viscosity enhancing agent that exhibits enhanced viscosity upon administration of the composition to an ocular surface of a human eye but then dissipates and gradually loses viscosity thereafter; and ii) thermally sensitive phase transition viscosity enhancing agent that exhibits a lower viscosity upon administration of the composition to the ocular surface of the human eye but then exhibits enhanced viscosity after administration to the ocular surface of the eye; and water.

In U.S. 2012/0269862 Chowhan et al. disclose a topical ophthalmic multi-dose aqueous composition comprising: a viscosity enhancing system comprised of: i) dissipation viscosity enhancing agent that exhibits enhanced viscosity upon administration of the composition to an ocular surface of a human eye but then dissipates and gradually loses viscosity thereafter; and ii) ion sensitive viscosity enhancing agent that exhibits a lower viscosity upon administration of the composition to the ocular surface of the human eye but then exhibits enhanced viscosity after administration to the ocular surface of the eye; and water.

In WO 2004/112836 Chowhan et al. disclose an aqueous composition suitable for topical ophthalmic administration comprising a viscosity enhancing amount of combination of two polymers having a synergistic effect on the composition's viscosity and wherein the combination of two polymers is selected from the group consisting of hydroxypropyl methylcellulose and guar gum; hydroxypropyl methylcellulose and a carboxyvinyl polymer; a carboxyvinyl polymer and guar gum; hydroxypropyl methylcellulose and hydroxyethylcellulose; hyaluronic acid and hydroxypropyl methylcellulose; and hyaluronic acid and guar gum, provided that if the composition comprises a carboxyvinyl polymer then the composition does not contain sodium chloride or boric acid.

In U.S. 2011/0117189 Mazzone et al. disclose a pharmaceutical composition for therapeutic use comprising xanthan gum as carrier of a therapeutically effective amount, sufficient for the treatment or prevention of pathologies of the posterior segment of the eye and in particular the retina, of an active principle selected from the group consisting of anti-infectives (antibiotics, antibacterials, antivirals, antifungals), steroidal and non-steroidal antiinflammatories, angiostatic cortisenes, COX inhibitors, antioxidants, angiogenesis inhibitors, neuroprotective agents, immunomodulating agents, vascular disrupting agents (VDA), immunosuppressant agents, antimetabolites and anti-VEGF.

There is still an unmet medical need for an ophthalmic formulation effective in the treatment of bacterial eye infections which provides prolonged residence time in the eye.

### BRIEF SUMMARY

This disclosure is directed to a composition comprising a gelled matrix in which particles of the active ingredient, e.g., fusidic acid, are suspended. This disclosure is also directed to the ophthalmic use of the composition for the treatment of bacterial eye infections.

The present disclosure is based in part on the unexpected finding of an ophthalmic formulation in which particles of the active ingredient are suspended in a gelled molecular matrix without the occurrence of sedimentation, flocculation and/or coagulation throughout the composition's shelf-life. The rheological properties of the gelled matrix, e.g., the yield stress at rest, assure that no particle sedimentation occurs even when subjecting the formulation to high acceleration (e.g., centrifugation of up to 10,000 rpm for 5 minutes). The formulation is therefore stable while affording prolonged residence time of the active ingredient in the eye.

According to a first aspect, there is provided an ophthalmic composition comprising: (a) a matrix comprising at least one non-ionic polymer and at least one anionic polymer, wherein the matrix is in the form of a gel, and (b) a particulate active ingredient suspended in said matrix, wherein the yield stress value of the composition at 25° C. is at least 5 Pa.

In one embodiment, the yield stress value of the composition at 25° C. is at least 10 Pa.

In some embodiments, the ratio between the non-ionic polymer and the anionic polymer in the composition is at least 1:1 (w/w). In various embodiments, the ratio between the non-ionic polymer and the anionic polymer in the composition is in the range of from about 1:1 to about 4:1 (w/w), including all iterations of ratios within the specified range. In other embodiments, the concentration of the non-ionic polymer in the composition is higher than the concentration of the anionic polymer.

In certain embodiments, the non-ionic polymer is selected from the group consisting of hydroxypropyl methylcellulose, hydroxypropyl cellulose, hydroxyethyl cellulose, hydroxymethyl cellulose, polyvinylpyrrolidone, methyl cellulose, polyvinyl alcohol, and a mixture thereof, with each possibility representing a separate embodiment. In one embodiment, the non-ionic polymer is hydroxypropyl methylcellulose. In particular embodiments, the hydroxypropyl methylcellulose (HPMC) is selected from the group consisting of HPMC K15M, HPMC K4M, and HPMC E4M, with each possibility representing a separate embodiment.

In some embodiments, the anionic polymer is selected from the group consisting of an anionic polysaccharide, an anionic gum, carboxymethylcellulose, and a mixture thereof, with each possibility representing a separate embodiment. In one embodiment, the anionic gum is xanthan gum.

In several embodiments, the active ingredient suspended in the matrix is an antibacterial agent. In particular embodiments, the antibacterial agent is antibiotics comprising at least one of fusidic acid, gentamicin, erythromycin, azithromycin, bacitracin, ciprofloxacin, polymyxin, doxycycline, cephalosporin, tobramycin, neomycin, ofloxacin, moxifloxacin, gatifloxacin, besifloxacin, chloramphenicol or a pharmaceutically acceptable salt thereof, and combinations thereof, with each possibility representing a separate embodiment. In one embodiment, the antibiotics is fusidic acid.

It will be recognized by one of skill in the art that the composition disclosed herein may further comprise an excipient such as an osmotic agent, a chelating agent, a buffering or a pH adjusting agent, a preservative, a wetting agent, a tonicity enhancing agent, or a combination or mixture thereof, with each possibility representing a separate embodiment.

In certain embodiments, the osmotic agent comprises at least one of mannitol, glycerol, sorbitol, xylitol, and combinations thereof. Each possibility represents a separate embodiment.

In various embodiments, the chelating agent comprises at least one of disodium edetate, deferoxamine mesylate (desferrioxamine), 2,3-dimercaprol, meso-2,3-dimercaptosuccinic acid (DMSA) and ester analogues thereof, deferiprone, nitrilotriacetic acid, and combinations thereof. Each possibility represents a separate embodiment.

In other embodiments, the buffering or pH adjusting agent comprises at least one of sodium hydroxide, potassium hydroxide, hydrochloric acid, and combinations thereof. Each possibility represents a separate embodiment.

In additional embodiments, the preservative comprises at least one of benzalkonium chloride, cetyl pyridinium chloride, parabens, benzoic acid, benzyl alcohol, thimerosal, phenylmercuric nitrate, chlorhexidine gluconate, chlorobutanol, and combinations thereof. Each possibility represents a separate embodiment.

In further embodiments, the wetting agent comprises at least one of benzododecinium bromide (BOB), cetrimide (Cet), and combinations thereof. Each possibility represents a separate embodiment.

In some embodiments, the tonicity enhancing agent comprises at least one of urea, glycerol, sorbitol, mannitol, propylene glycol, dextrose, and combinations thereof. Each possibility represents a separate embodiment.

There is further provided a method of preparing an ophthalmic composition as disclosed herein, the method comprising the steps of: (a) dissolving at least one non-ionic polymer and at least one anionic polymer in an aqueous medium optionally comprising a pharmaceutically acceptable excipient; (b) inducing gelation of the at least one non-ionic polymer and at least one anionic polymer thereby forming a matrix in the form of a gel; and (c) suspending a particulate active ingredient in the thus obtained gelled-matrix.

According to additional embodiments, the ophthalmic composition as disclosed herein is useful for the treatment of a bacterial eye infection (e.g., bacterial conjunctivitis). In some embodiments, there is provided a method of treating a bacterial eye infection in a subject in need thereof, the method comprising topically administering an effective amount of the composition disclosed herein to the subject's eye.

In certain embodiments, the bacteria are gram positive bacteria. In other embodiments, the subject is a mammal, preferably a human.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows shear stress (SS) vs. shear rate (SR) (solid line) of a formulation containing 1% hyaluronic acid 300 in which 1% fusidic acid is suspended. Linear fitting (dotted line) and extrapolation for the intersection with the y-axis indicates a yield stress point of 0.0829 Pa.

FIG. 2 shows shear stress (SS) vs. shear rate (SR) (solid line) of a formulation containing 0.5% HPMC K4M and 1% hyaluronic acid 300 in which 1% fusidic acid is suspended. Linear fitting (dotted line) and extrapolation for the intersection with the y-axis indicates a yield stress point of 0.6291 Pa.

FIG. 3 shows shear stress (SS) vs. shear rate (SR) (solid line) of a formulation containing 0.5% HPMC K15M and 1% hyaluronic acid 300 in which 1% fusidic acid is suspended. Linear fitting (dotted line) and extrapolation for the intersection with the y-axis indicates a yield stress point of 2.7543 Pa.

FIG. 4 shows shear stress (SS) vs. shear rate (SR) (solid line) of a formulation containing 1.5% HPMC E4M in which 1% fusidic acid is suspended. Linear fitting (dotted line) and extrapolation for the intersection with the y-axis indicates a yield stress point of 1.4044 Pa.

FIG. 5 shows shear stress (SS) vs. shear rate (SR) (solid line) of a formulation containing 0.5% HPMC E4M in which 1% fusidic acid is suspended. Linear fitting (dotted line) and extrapolation for the intersection with the y-axis indicates a yield stress point of 0.0478 Pa.

FIG. 6 shows shear stress (SS) vs. shear rate (SR) (solid line) of a formulation containing 0.5% HPMC E4M and 1% hyaluronic acid 300 in which 1% fusidic acid is suspended. Linear fitting (dotted line) and extrapolation for the intersection with the y-axis indicates a yield stress point of 0.6968 Pa.

FIG. 7 shows shear stress (SS) vs. shear rate (SR) (solid line) of a formulation containing 1.2% HPMC K15M and 0.5% xanthan gum in which 1% fusidic acid is suspended. Linear fitting (dotted line) and extrapolation for the intersection with the y-axis indicates a yield stress point of 10.907 Pa.

FIG. 8 shows shear stress (SS) vs. shear rate (SR) (solid line) of a formulation containing 1.7% HPMC K4M and 0.5% xanthan gum in which 1% fusidic acid is suspended. Linear fitting (dotted line) and extrapolation for the intersection with the y-axis indicates a yield stress point of 11.242 Pa.

FIG. 9 shows shear stress (SS) vs. shear rate (SR) (solid line) of a formulation containing 1.7% HPMC E4M and 0.5% xanthan gum in which 1% fusidic acid is suspended. Linear fitting (dotted line) and extrapolation for the intersection with the y-axis indicates a yield stress point of 10.19 Pa.

### DETAILED DESCRIPTION

The present disclosure provides an ophthalmic composition comprising a matrix in the form of a gel and a particulate active agent suspended therein. The composition is designed to provide prolonged residence time of the active agent in the eye. Methods of preparing said composition and use thereof are disclosed as well.

Using matrices in the form of a gel enables the suspension of particles of a wide variety of active agents suitable for use in ophthalmic formulations. Exemplary active agents that may be incorporated into the composition of the disclosure include, but are not limited to, steroids such as dexamethasone, prednisolone, fluorometholone, loteprednol and the like; antiviral agents such as acyclovir; non-steroidal anti-inflammatory drugs such as indomethacin, diclofenac, nepafenac and the like; anti-allergy agents such as ketotifen, lodoxamide and the like; and antibacterial agents such as fusidic acid, gentamicin, erythromycin, azithromycin, bacitracin, ciprofloxacin, polymyxin, doxycycline, cephalosporin, tobramycin, neomycin, ofloxacin, moxifloxacin, gatifloxacin, besifloxacin, chloramphenicol and the like or pharmaceutically acceptable salts of any of the aforementioned active agents. It is contemplated that these active agents have miscibility characteristics that enable their incorporation into the composition as particulate material. In some embodiments, the active agents suitable for being incorporated into the composition disclosed herein exhibit maximum solubility of about 2% in the gelled matrix.

According to the principles disclosed herein, the matrix is characterized by having a yield stress sufficient to prevent sedimentation, flocculation and/or coagulation of the particles of the active agent thereby affording long-term stability. Moreover, contrary to liquid compositions which upon eye administration are typically washed/diluted (by blinking and tear fluid) to about 15% after 10 minutes with drug absorption typically occurring 2-3 minutes after administration, the composition of the present disclosure is in the form of a gelled matrix thereby enabling prolonged residence time of the active agent suspended therein in the eye. It is contemplated that the prolonged residence time does not result in from in-situ gel formation but is rather afforded by the molecular structure of the gelled matrix composition prior to administration such that no thickening or further gelation of the composition occurs under physiological conditions.

According to the principles disclosed herein, the matrix provides a gelatinous medium suitable for supporting suspended particles of the active agent. In certain embodiments, the gelled-matrix comprises a continuous skeleton formed by a combination of two or more gel-forming polymers in which the particulate matter (i.e. the active agent) is suspended. It is believed that the structure of the gel-forming polymers in the composition resembles a molecular sieve structure having pores in the sub-micrometer range thereby preventing sedimentation of a micronized particulate matter.

The matrix, according to the principles disclosed herein, is characterized by a yield stress value at 25° C. of at least 5 Pa. A yield stress value or yield point refers to the stress at which a material begins to deform plastically. When applying stress values that are lower than the yield point, the material deforms elastically thereby returning to its original shape when the applied stress is removed. Once a stress that exceeds the yield point is applied to a material, at least a fraction of the deformation of the material is permanent and non-reversible. According to certain embodiments of the disclosure, physical stability is afforded by a composition characterized by a yield stress value at rest (shear rate extrapolated to zero) of at least 5 Pa. According to other embodiments of the disclosure, the composition is characterized by a yield stress value at rest (shear rate extrapolated to zero) of at least 10 Pa. The yield stress values at 25° C. are typically in the range of about 5 to about 50 Pa, preferably in the range of about 5 to about 20 Pa, including each integer within the specified range. For example, the yield stress value of the ophthalmic composition disclosed herein at 25° C. is about 5, about 6, about 7, about 8, about 9, about 10, about 11, about 12, about 13, about 14, about 15, about 16, about 17, about 18, about 19, about 20, about 22, about 25, about 28, about 30, about 32, about 35, about 38, about 40, about 42, about 45, about 48, or about 50 Pa. Each possibility represents a separate embodiment.

The yield stress value may be determined using various methods and techniques known to those skilled in the art. For example, the yield stress value may be determined using direct rheological measurements (e.g., using a controlled stress rheometer). In certain embodiments, the yield stress value may be determined using various curve fitting models including, but not limited to, linear regression, Ostwald de Wade, and Bingham. Each possibility represents a separate embodiment. In other embodiments, the yield stress value may be determined by linear fitting of the shear stress vs. shear rate at shear rates of about 0.05 to about 1,000 sec⁻¹, for example about 1 to about 100 sec⁻¹, about 100 to about 1,000 sec⁻¹, or about 0.05 to about 25 sec⁻¹ and the like, including each integer within the specified range, and extrapolation to zero shear rate (intersection with the y-axis). Each possibility represents a separate embodiment.

The polymers in the gelled-matrix may be selected to afford the desired rheological characteristics. Parameters that may influence the selection of polymers include, but are not limited to, viscosity, gel strength etc. The polymers may initially be in a gelatin-like state or may be gelled using a gelling agent as is known in the art.

According to the principles disclosed herein, the matrix comprises a polymer combination. The gel formed by the combination of gel-forming polymers enables the manipulation of the properties of the resultant gel in order to provide the desired rheological properties, and in particular the yield stress. Typically, the polymer combination comprises a combination of a non-ionic polymer and an anionic polymer.

Non-limiting examples of non-ionic polymers which are suitable for use in embodiments according to the principles presented herein include hydroxypropyl methylcellulose (HPMC), mixtures of hydroxypropyl methylcellulose polymers, hydroxypropyl cellulose (HPC), mixtures of hydroxypropyl cellulose polymers, hydroxyethyl cellulose, mixtures of hydroxyethyl cellulose polymers, hydroxymethyl cellulose, mixtures of hydroxymethyl cellulose polymers, polyvinylpyrrolidone (povidone, PVP), mixtures of polyvinylpyrrolidone polymers, methyl cellulose, mixture of methyl cellulose polymers, polyvinyl alcohol, mixtures of polyvinyl alcohol polymers, and combinations thereof, with each possibility representing a separate embodiment. In one embodiment, the non-ionic polymer is HPMC. It is contemplated that different grades of HPMC such as, but not limited to, K15M, K4M, E4M and combinations thereof, are included within the scope of the present disclosure. In one embodiment, the non-ionic polymer is hydroxypropyl methylcellulose (HPMC) having a viscosity of a 2% aqueous solution at 20° C. which is about 1,000 to about 6,000 cP, preferably about 2,000 to about 5,000 cP, including each integer within the specified range. In another embodiment, the non-ionic polymer is hydroxypropyl methylcellulose (HPMC) having a viscosity of a 2% aqueous solution at 20° C. which is about 11,000 to about 22,000 cP, including each integer within the specified range.

According to certain aspects and embodiments, the amount of the non-ionic polymer in the composition is in the range of from about 0.1% to about 10% by weight of the total ophthalmic composition mass, including each integer within the specified range. Preferably, the amount of the non-ionic polymer in the composition is in the range of from about 0.2% to about 3% by weight of the total ophthalmic composition mass, including each integer within the specified range. In one embodiment, the amount of the non-ionic polymer in the composition is about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the total ophthalmic composition mass, with each possibility representing a separate embodiment. In another embodiment, the amount of the non-ionic polymer in the composition is about 1.2% by weight of the total ophthalmic composition mass. In yet another embodiment, the amount of the non-ionic polymer in the composition is about 1.7% by weight of the total ophthalmic composition mass.

Non-limiting examples of anionic polymers which are suitable for use in embodiments according to the principles presented herein include anionic polysaccharides or a mixture of anionic polysaccharide polymers and carboxy polysaccharides or a mixture of carboxy polysaccharide polymers such as sodium alginate, alginic acid, pectin, hyaluronic acid, polyglucuronic acid (poly-α- and -β-1,4-glucuronic acid), polygalacturonic acid (pectic acid), chondroitin sulfate, carrageenan, furcellaran, and carboxymethylcellulose, with each possibility representing a separate embodiment; anionic gums such as xanthan gum, and the like or a mixture of anionic gum polymers. In one embodiment, the anionic polymer is xanthan gum.

According to certain aspects and embodiments, the amount of the anionic polymer in the composition is in the range of from about 0.01% to about 4% by weight of the total ophthalmic composition mass, including each integer within the specified range. Preferably, the amount of the anionic polymer in the composition is in the range of from about 0.1% to about 2% by weight of the total ophthalmic composition mass, including each integer within the specified range. In one embodiment, the amount of the anionic polymer in the composition is about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, or about 4% by weight of the total ophthalmic composition mass, with each possibility representing a separate embodiment. In another embodiment, the amount of the anionic polymer in the composition is about 0.5% by weight of the total ophthalmic composition mass.

The ratio between the non-ionic polymer and the anionic polymer can be adjusted to provide adequate physical stability to the composition. Typically, the ratio is in the range of from about 1:1 to about 4:1 (w/w), including all iterations of ratios within the specified range. For example, the ratio between the non-ionic polymer and the anionic polymer in the ophthalmic composition disclosed herein is about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.9:1, or about 4:1 (w/w). Each possibility represents a separate embodiment. In one embodiment, the ratio between the non-ionic polymer and the anionic polymer in the ophthalmic composition disclosed herein is about 2.4:1 (w/w). In another embodiment, the ratio between the non-ionic polymer and the anionic polymer in the ophthalmic composition disclosed herein is about 3.4:1 (w/w).

According to some embodiments, the active ingredient which is suspended in the gelled matrix is an antibacterial agent. Suitable antibacterial agents include, but are not limited to, antibiotics such as polypeptide antibiotic, aminoglycoside antibiotic, macrolide antibiotic, antifolate antibiotic, quinolone antibiotic, sulfa antibiotic, tetracycline antibiotic and the like. Each possibility represents a separate embodiment. Exemplary antibacterial agents include, but are not limited to, fusidic acid, gentamicin, erythromycin, azithromycin, bacitracin, ciprofloxacin, polymyxin, doxycycline, cephalosporin, tobramycin, neomycin, ofloxacin, moxifloxacin, gatifloxacin, besifloxacin, chloramphenicol or a pharmaceutically acceptable salt thereof, and mixtures thereof. Each possibility represents a separate embodiment. It is contemplated that the aforementioned antibacterial agents are incorporated into the composition in the form of particles thereby exhibiting maximum solubility of about 2% in the gelled matrix.

A currently preferred antibacterial agent is fusidic acid. Fusidic acid is a bacteriostatic antibiotic suitable for the treatment of bacterial infections caused by e.g. Staphylococcus aureus, Streptococcus pneumonia, and Haemophilus influenzae. The global problem of advancing antimicrobial resistance has led to a renewed interest in the use of fusidic acid. Advantageously, the composition of the present disclosure is in the form of a gelled-matrix in which particles of active ingredient are suspended (rather than being dissolved) thereby allowing the use of active agents such as fusidic acid which are substantially immiscible in aqueous media.

According to certain aspects and embodiments, the amount of the active agent in the composition is in the range of from about 0.1% to about 2% by weight of the total ophthalmic composition mass, including each integer within the specified range. Preferably, the amount of the active agent in the composition is in the range of from about 0.5% to about 1.5% by weight of the total ophthalmic composition mass, including each integer within the specified range. In one embodiment, the amount of the active agent in the composition is about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% by weight of the total ophthalmic composition mass, with each possibility representing a separate embodiment. In another embodiment, the amount of the active agent in the composition is about 1% by weight of the total ophthalmic composition mass.

The composition disclosed herein may optionally comprise at least one excipient, such as an osmotic agent, a chelating agent, a buffering or a pH adjusting agent, a preservative, a wetting agent, a tonicity enhancing agent, or a combination or mixture thereof. Each possibility represents a separate embodiment.

Suitable osmotic agents that may be incorporated into the ophthalmic composition include, but are not limited to, mannitol, glycerol, sorbitol, xylitol and combinations thereof, with each possibility representing a separate embodiment.

Suitable chelating agents that may be incorporated into the ophthalmic composition include, but are not limited to, disodium edetate, deferoxamine mesylate (desferrioxamine), 2,3-dimercaprol, meso-2,3-dimercaptosuccinic acid (DMSA) and its ester analogues, deferiprone, nitrilotriacetic acid (NTA) and combinations thereof, with each possibility representing a separate embodiment. In one embodiment, the chelating agent comprises disodium edetate. In another embodiment, the amount of the chelating agent in the composition is in the range of from about 0.001% to about 1% by weight of the total ophthalmic composition mass. In certain embodiments, the amount of the chelating agent in the composition is about 0.001%, about 0.005%, about 0.01%, about 0.015%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.08%, about 0.085%, about 0.09%, about 0.095%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1% by weight of the total ophthalmic composition mass, with each possibility representing a separate embodiment. In one embodiment, the amount of the chelating agent in the composition is about 0.05% by weight of the total ophthalmic composition mass.

Suitable buffering or pH adjusting agents that may be incorporated into the ophthalmic composition include, but are not limited to, sodium hydroxide, potassium hydroxide, hydrochloric acid and combinations thereof, with each possibility representing a separate embodiment. The buffering or pH adjusting agents are typically incorporated into the ophthalmic composition in amounts suitable for obtaining a pH in the range of from about 3.5 to about 8.5, including each integer within the specified range. In one embodiment, the buffering or pH adjusting agents are incorporated into the ophthalmic composition in amounts suitable for obtaining a pH in the range of from about 5 to about 6.

Suitable preservatives that may be incorporated into the ophthalmic composition include, but are not limited to, benzalkonium chloride, cetyl pyridinium chloride, parabens, benzoic acid, benzyl alcohol, thimerosal, phenylmercuric nitrate, chlorhexidine gluconate, chlorobutanol and combinations thereof, with each possibility representing a separate embodiment. In one embodiment, the preservative comprises benzalkonium chloride. In another embodiment, the amount of the preservative in the composition is in the range of from about 0.001% to about 1% by weight of the total ophthalmic composition mass. In certain embodiments, the amount of the preservative in the composition is about 0.001%, about 0.002%, about 0.003%, about 0.004%, about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1% by weight of the total ophthalmic composition mass, with each possibility representing a separate embodiment. In one embodiment, the amount of the preservative in the composition is about 0.01% by weight of the total ophthalmic composition mass.

Suitable wetting agents that may be incorporated into the ophthalmic composition include, but are not limited to, benzododecinium bromide (BOB), cetrimide (Cet), and combinations or mixtures thereof, with each possibility representing a separate embodiment.

Suitable tonicity enhancing agents that may be incorporated into the ophthalmic composition include ionic and non-ionic agents. For example, ionic compounds include, but are not limited to, alkali metal or alkaline earth metal halides, such as, for example, CaCl₂ KBr, KCl, LiCl, NaI, NaBr or NaCl, and boric acid or a mixture or combination thereof, with each possibility representing a separate embodiment. Non-ionic tonicity enhancing agents are, for example, urea, glycerol, sorbitol, mannitol, propylene glycol, and dextrose or a mixture or combination thereof, with each possibility representing a separate embodiment. In currently preferred embodiments, the tonicity enhancing agent is a non-ionic tonicity enhancing agent. In one embodiment, the tonicity enhancing agent comprises mannitol. In another embodiment, the amount of the tonicity enhancing agent in the composition is in the range of from about 0.5% to about 15% by weight of the total ophthalmic composition mass, including each integer within the specified range. In certain embodiments, the amount of the tonicity enhancing agent in the composition is about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight of the total ophthalmic composition mass, with each possibility representing a separate embodiment. In one embodiment, the amount of the tonicity enhancing agent in the composition is about 4.5% by weight of the total ophthalmic composition mass.

The ophthalmic composition of the present disclosure can be prepared by any method known to those skilled in the art. In some embodiments, the ophthalmic composition is prepared using low and/or high shear mixers such as, but not limited to, shaker mixers, propeller mixers, turbine mixers, paddle mixers, rotor-stator mixers, homogenizers, colloid mill, and the like. Each possibility represents a separate embodiment. Typically, the gel-forming polymers are dissolved in an aqueous medium which optionally comprises excipient(s). Optionally, the aqueous medium is heated in order to afford dissolution of the gel-forming polymers. Gelling is then induced either physically (e.g. via cooling) or chemically (via the addition of a gelling agent) followed by admixing of the active agent in particulate form. Within the scope of the present disclosure are particles of the active agent which are characterized by a diameter in the micrometer range. Preferably, the particles have diameters of 10 μm or less so as to avoid damage to the cornea upon administration. Exemplary diameters include, but are not limited to, about 0.1 μm to about 9 μm, preferably about 0.5 μm to about 8 μm, and more preferably about 1 μm to about 7 μm, including each integer within the specified range. The admixture of the active agent with the gelled matrix is performed so as to afford a substantially uniform distribution of the particles of active agent in the gel matrix. Optionally, an additional step of pH adjustment, typically to a value of about 3.5 to about 8.5, preferably about 5 to about 6, is performed. Sterilization may be effected to each of the components separately or some of the components combined, followed by the assembly of the components as described herein under aseptic conditions to obtain the composition of the disclosure. This is mainly performed when one or more components of the composition are sensitive to a single method of sterilization thereby requiring the use of several methods of sterilization. Alternatively, sterilization may be effected to the entire composition. Suitable sterilization methods include, but are not limited to, autoclaving, gamma irradiation, and the like. Each possibility represents a separate embodiment.

In certain embodiments, the ophthalmic composition is useful for treating bacterial eye infection or bacterial conjunctivitis.

Accordingly, there is provided a method of treating a bacterial eye infection such as bacterial conjunctivitis, the method comprising topically administering a therapeutically effective amount of the composition disclosed herein to the eye of a subject in need thereof. The subject in need thereof is typically a mammal, preferably a human.

The ophthalmic composition may be applied directly to the eye, the eye lid, or other tissue surrounding the eye. For example, the composition may be applied to the fornix inferior of the infected eye to afford local treatment of the bacterial eye infection or bacterial conjunctivitis.

The term “therapeutically effective amount” or “an effective amount” as used herein refers to a quantity of a compound which is sufficient to provide a beneficial effect to the subject to which the compound is administered. The effective amount, according to the principles disclosed herein, can be determined by any one of ordinary skill in the art and can be tested on various models both in-vitro and in-vivo.

The effective amount of a composition to be administered as well as the administration regimen depends on various factors including the subject being treated (e.g., the subject's age) and the severity of the disease, and can be determined by the judgment of the prescribing physician. Because of patient-to-patient variability, dosages are a guideline only and the physician may adjust doses of the compounds and compositions to achieve the level of effective treatment that the physician considers appropriate for the patient. In considering the degree of treatment desired, the physician must balance a variety of factors such as the age of the patient and the presence of other diseases or conditions.

The term “treating” as used herein refers to stopping or slowing down the progression of the disease. The term “treating” further includes the reduction in the occurrence of various symptoms associated with a bacterial eye infection. In one embodiment, treating comprises the inhibition of bacterial replication accompanied by the reduction of bacterial load. In other embodiments, treating comprises essentially complete eradication of the bacteria.

Various bacteria are known to be sensitive to fusidic acid, particularly gram positive bacteria. Exemplary bacteria include, but are not limited to, Staphylococcus aureus, Coagulase-negative staphylococci, Streptococcus pneumonia, Streptococcus Viridans, Haemophilus influenza, Moraxella catarrhali, and Corynebacterium species. Additional bacteria which are sensitive to fusidic acid include, but are not limited to, Micrococcus luteus, Clostridium species, Peptostreptococcus species, gram-negative non-fermentative bacilli, and Neisseria species. It is contemplated that the composition disclosed herein may be used to stopping or slowing down the progression of eye infection or bacterial conjunctivitis which is induced by any of the aforementioned bacteria.

As used herein and in the appended claims, the term “about” refers to ±10%.

As used herein and in the appended claims, the singular forms “a”, “an”, and “the” include plural references unless the context clearly dictates otherwise.

It should be noted that the term “and” or the term “or” are generally employed in its sense including “and/or” unless the context clearly dictates otherwise.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

An ophthalmic composition according to certain embodiments of the present disclosure was prepared as follows: A solution containing benzalkonium chloride, disodium edetate and mannitol in water for injection was heated to about 80° C. HPMC (methocel) and xanthan gum or HPMC (methocel) and hyaluronic acid at ratios of about 1:1 to about 4:1 (w/w) were then added to obtain a clear solution. The solution was then cooled to room temperatures to induce gelation of the polymers followed by the addition of fusidic acid particles having diameters of less than 10 μm. The particles were mixed and a homogenous suspension was obtained. Optionally, 5N NaOH was added to adjust the pH to 5-6.

Exemplary formulations are outlined in Tables 1-3 below:

TABLE 1

| Substance | mg |
|---|---|
| Fusidic acid | 10 |
| Methocel Kl5M | 12 |
| Xanthan gum | 5 |
| Mannitol | 45 |
| Disodium edetate | 0.5 |
| Benzalkonium chloride | 0.1 |
| Water for injection up to | 1.00 g |
| NaOH 5N | q.s. to a pH of 5-6 |

TABLE 2

| Substance | mg |
|---|---|
| Fusidic acid | 10 |
| Methocel K4M | 17 |
| Xanthan gum | 5 |
| Mannitol | 45 |
| Disodium edetate | 0.5 |
| Benzalkonium chloride | 0.1 |
| Water for injection up to | 1.00 g |
| NaOH 5N | q.s. to a pH of 5-6 |

TABLE 3

| Substance | mg |
|---|---|
| Fusidic acid | 10 |
| Methocel E4M | 17 |
| Xanthan gum | 5 |
| Mannitol | 45 |
| Disodium edetate | 0.5 |
| Benzalkonium chloride | 0.1 |
| Water for injection up to | 1.00 g |
| NaOH 5N | q.s. to a pH of 5-6 |

### Example 2

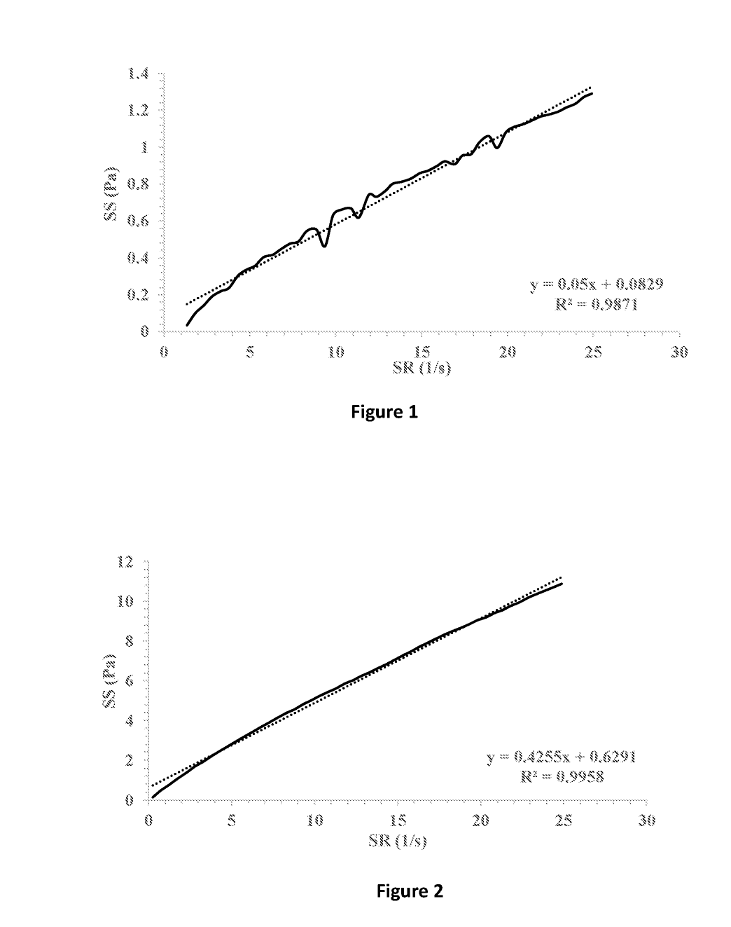

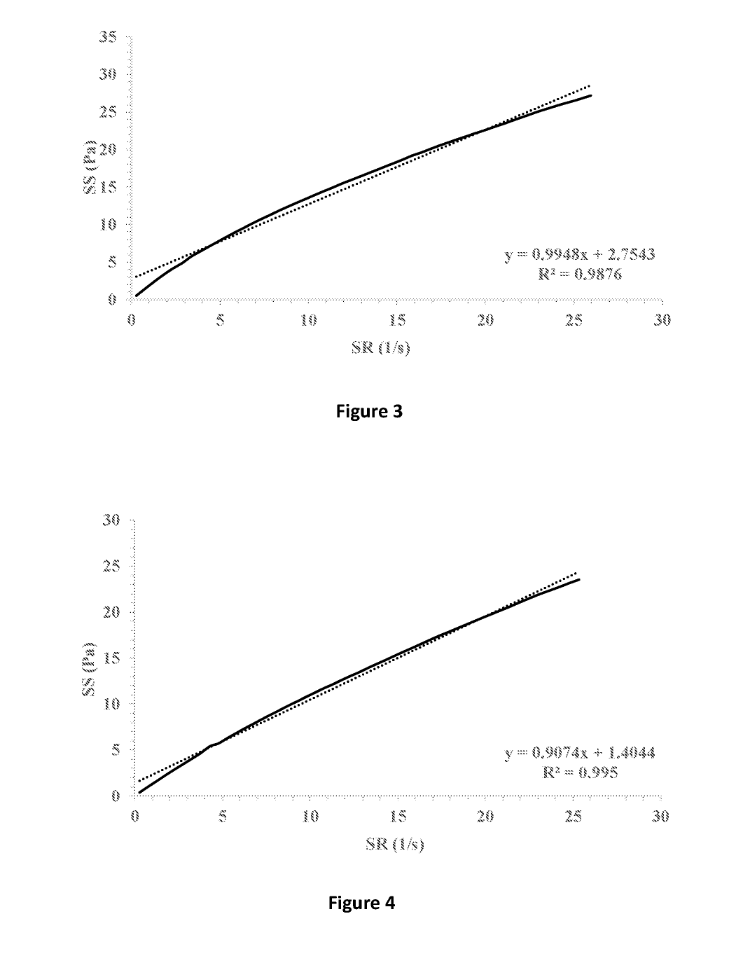

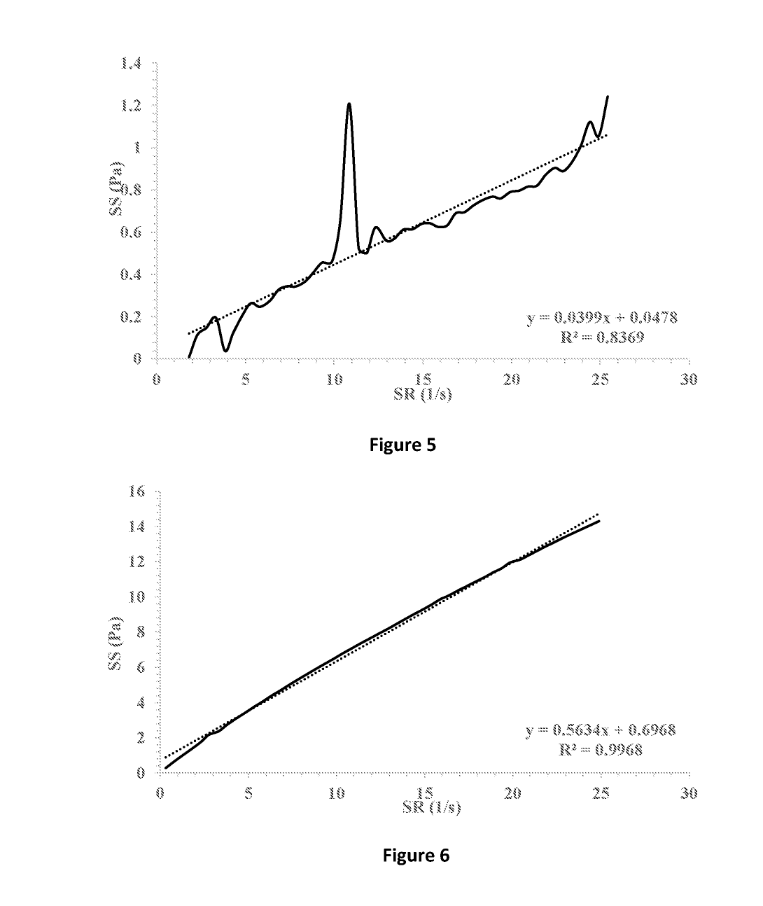

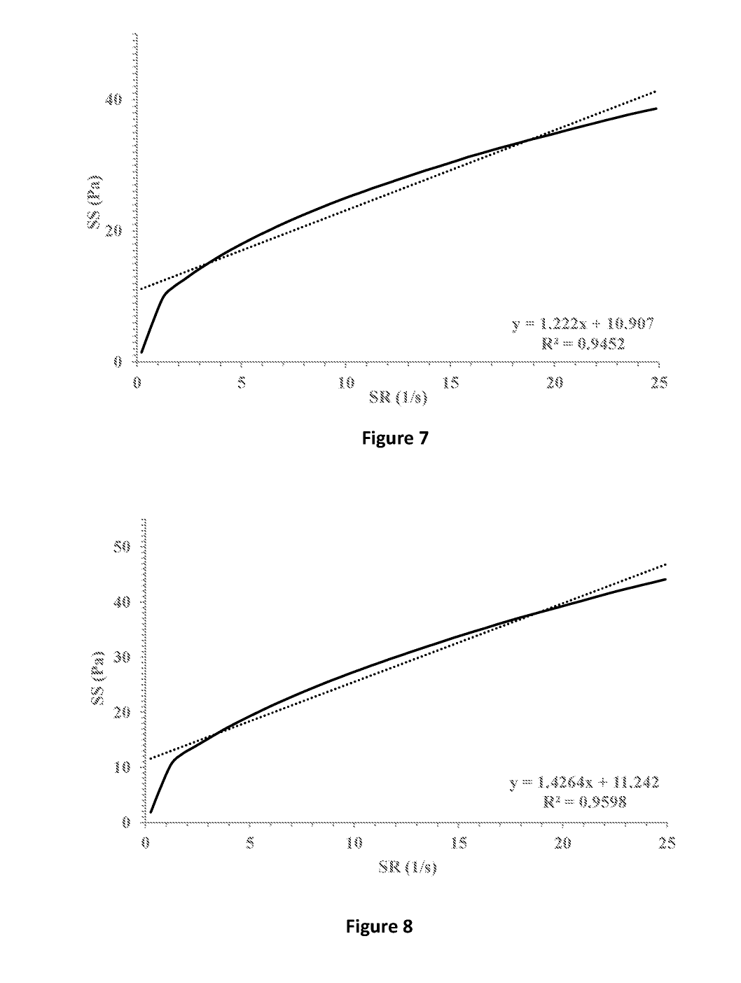

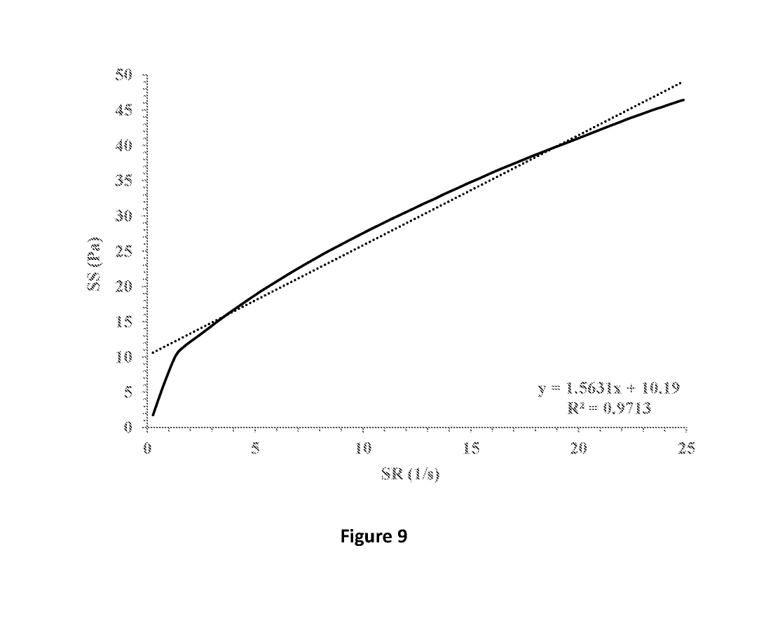

The ophthalmic compositions as set forth in Tables 1-3 were evaluated for their yield values at low shear rates. The yield values were assessed using shear rate vs. shear stress measurements and extrapolation of the linear fitting of the curves for the intersection with the y-axis (FIGS. 7-9, respectively). The following compositions were used as control: 1% fusidic acid suspended in 1% hyaluronic acid 300 (designated HA), FIG. 1; 1% fusidic acid suspended in a mixture of 1% hyaluronic acid 300 and 0.5% HPMC (methocel K4M), FIG. 2; 1% fusidic acid suspended in a mixture of 1% hyaluronic acid 300 and 0.5% HPMC (methocel K15M), FIG. 3; 1% fusidic acid suspended in 1.5% HPMC (methocel E4M), FIG. 4; 1% fusidic acid suspended in 0.5% HPMC (methocel E4M), FIGS. 5; and 1% fusidic acid suspended in a mixture of 1% hyaluronic acid 300 and 0.5% HPMC (methocel E4M), FIG. 6. Whereas the yield stress values of control samples are in the range of 0.04-2.75 Pa, the yield stress values of the ophthalmic compositions as set forth in Tables 1-3 all exceed 5 Pa.

### Example 3

In order to assess the physical stability of the ophthalmic compositions according to certain embodiments of the disclosure, exemplary compositions were subjected to accelerated centrifugal conditions of 100-4,000 rounds per minute (rpm) for a total of 2 minutes. Compositions containing 0.5% xanthan gum (designated XG) and 1-2% HPMC of different grades (designated MC K15M, MC E4M, and MC K4M) according to certain embodiments of the disclosure were compared to the control compositions detailed in Example 2. Table 4 summarizes the results where V and X indicate no sedimentation and sedimentation of the fusidic acid particles, respectively.

TABLE 4

| | | | | | | | | | | | |
|---|---|---|---|---|---|---|---|---|---|---|---|
| Description | 100 rpm | 300 rpm | 500 rpm | 700 rpm | 1000 rpm | 1300 rpm | 1600 rpm | 2000 rpm | 2500 rpm | 3000 rpm | 4000 rpm |

Compositions according to certain embodiments of the disclosure

| | | | | | | | | | | | |
|---|---|---|---|---|---|---|---|---|---|---|---|
| 0.5% XG + | v | v | v | v | v | v | v | v | v | v | v |
| 1% MC | | | | | | | | | | | |
| K15M | | | | | | | | | | | |
| 0.5% XG + | v | v | v | v | v | v | v | v | v | v | v |
| 1.5% MC | | | | | | | | | | | |
| K15M | | | | | | | | | | | |
| 0.5% XG + | v | v | v | v | v | v | v | v | v | v | v |
| 1.5% MC | | | | | | | | | | | |
| K4M | | | | | | | | | | | |
| 0.5% XG + | v | v | v | v | v | v | v | v | v | v | v |
| 2% MC | | | | | | | | | | | |
| K4M | | | | | | | | | | | |
| 0.5% XG + | v | v | v | v | v | v | v | v | v | v | v |
| 1.5% MC | | | | | | | | | | | |
| E4M | | | | | | | | | | | |
| 0.5% XG + | v | v | v | v | v | v | v | v | v | v | v |
| 2% MC | | | | | | | | | | | |
| E4M | | | | | | | | | | | |

Controls

| | | | | | | | | | | | |
|---|---|---|---|---|---|---|---|---|---|---|---|
| 1% HA | v | x | x | x | x | x | x | x | x | x | x |
| 1% HA300 + | v | v | v | v | v | v | v | v | x | x | x |
| 0.5% MC | | | | | | | | | | | |
| K4M | | | | | | | | | | | |
| 1% HA + | v | v | v | v | v | v | v | v | v | x | x |
| 0.5% MC | | | | | | | | | | | |
| K15M | | | | | | | | | | | |
| 1.5% MC | v | v | v | v | v | v | v | v | v | x | x |
| E4M | | | | | | | | | | | |
| 0.5% MC | v | x | x | x | x | x | x | x | x | x | x |
| E4M | | | | | | | | | | | |
| 1% HA + | v | v | v | v | v | v | v | v | x | x | x |
| 0.5% MC | | | | | | | | | | | |
| E4M | | | | | | | | | | | |

Table 4 clearly shows that compositions having yield values that exceed 5 Pa show no sedimentation of the fusidic acid particles even at high acceleration, whereas sedimentation does occur in control compositions having lower yield values. Accordingly, the compositions according to certain embodiments of the present disclosure showed physical stability even under high acceleration conditions. Thus, compositions characterized by a yield value of at least 5 Pa are physically stable and show no sedimentation of the fusidic acid particles suspended therein.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. An ophthalmic composition comprising:
(a) a matrix comprising at least one non-ionic polymer and at least one anionic polymer, wherein the matrix is in the form of a gel; and
(b) a particulate active ingredient suspended in said matrix,
wherein the yield stress value of the composition at 25° C. is at least 5 Pa.

2. The ophthalmic composition of claim 1, wherein the yield stress value of the composition at 25° C. is at least 10 Pa.

3. The ophthalmic composition of claim 1 or 2, wherein the ratio between the non-ionic polymer and the anionic polymer in the composition is in the range of from about 1:1 to about 4:1 (w/w).

4. The ophthalmic composition of claim 1, wherein the non-ionic polymer is selected from the group consisting of hydroxypropyl methylcellulose, hydroxypropyl cellulose, hydroxyethyl cellulose, hydroxymethyl cellulose, polyvinylpyrrolidone, methyl cellulose, polyvinyl alcohol, and a mixture thereof.

5. The ophthalmic composition of claim 4, wherein the non-ionic polymer is hydroxypropyl methylcellulose.

6. The ophthalmic composition of claim 5, wherein the hydroxypropyl methylcellulose (HPMC) is selected from the group consisting of HPMC K15M, HPMC K4M, and HPMC E4M.

7. The ophthalmic composition of claim 1, wherein the anionic polymer is selected from the group consisting of an anionic polysaccharide, an anionic gum, and a mixture thereof.

8. The ophthalmic composition of claim 7, wherein the anionic gum is xanthan gum.

9. The ophthalmic composition of claim 1, wherein the active ingredient is an antibacterial agent.

10. The ophthalmic composition of claim 9, wherein the antibacterial agent is antibiotics comprising at least one of fusidic acid, gentamicin, erythromycin, azithromycin, bacitracin, ciprofloxacin, polymyxin, doxycycline, cephalosporin, tobramycin, neomycin, ofloxacin, moxifloxacin, gatifloxacin, besifloxacin, chloramphenicol or a pharmaceutically acceptable salt thereof and a combination thereof.

11. The ophthalmic composition of claim 10, wherein the antibiotics is fusidic acid.

12. The ophthalmic composition of claim 1 further comprising an excipient selected from the group consisting of an osmotic agent, a chelating agent, a buffering or a pH adjusting agent, a preservative, a wetting agent, a tonicity enhancing agent, and a combination thereof.

13. The ophthalmic composition of claim 12, wherein the osmotic agent comprises at least one of mannitol, glycerol, sorbitol, xylitol, and combinations thereof.

14. The ophthalmic composition of claim 12, wherein the chelating agent comprises at least one of disodium edetate, deferoxamine mesylate (desferrioxamine), 2,3-dimercaprol, meso-2,3-dimercaptosuccinic acid (DMSA) and ester analogues thereof, deferiprone, nitrilotriacetic acid, and combinations thereof.

15. The ophthalmic composition of claim 12, wherein the buffering or pH adjusting agent comprises at least one of sodium hydroxide, potassium hydroxide, hydrochloric acid, and combinations thereof.

16. The ophthalmic composition of claim 12, wherein the preservative comprises at least one of benzalkonium chloride, cetyl pyridinium chloride, parabens, benzoic acid, benzyl alcohol, thimerosal, phenylmercuric nitrate, chlorhexidine gluconate, chlorobutanol, and combinations thereof.

17. The ophthalmic composition of claim 12, wherein the wetting agent comprises at least one of benzododecinium bromide (BOB), cetrimide (Cet), and combinations thereof.

18. The ophthalmic composition of claim 12, wherein the tonicity enhancing agent comprises at least one of urea, glycerol, sorbitol, mannitol, propylene glycol, dextrose, and combinations thereof.

19. A method of preparing the ophthalmic composition of claim 1, comprising the steps of:
(a) dissolving at least one non-ionic polymer and at least one anionic polymer in an aqueous medium optionally comprising a pharmaceutically acceptable excipient;
(b) inducing gelation of the at least one non-ionic polymer and at least one anionic polymer thereby forming a matrix in the form of a gel; and
(c) suspending a particulate active ingredient in the gelled-matrix.

20. An ophthalmic composition prepared according to the method of claim 19.

21. (canceled)

22. A method of treating a bacterial eye infection in a subject in need thereof, the method comprising topically administering an effective amount of the ophthalmic composition of claim 1 to the subject's eye.
